(19) United States
(12) Patent Application Publication
(10) Pub. No.: US 2005/0182436 A1
(43) Pub. Date: Aug. 18, 2005

(54) Apparatus and method for creating working channel through tissue

(75) Inventor: Gopal K. Chopra, San Francisco, CA (US)

Correspondence Address: Bingham McCuthen, LLP;Suite 1800, Three Embarcadero, San Francisco, CA, 94111-4067, US

(73) Assignee: Scimed Life Systems, Inc., Maple Grove, MN (US)

(21) Appl. No.: 10/782,359

(22) Filed: Feb. 18, 2004

(51) Int. Cl. A61M 29/02; A61F 2/06

(52) U.S. Cl. 606/192; 623/1.11

(57) ABSTRACT

A minimally invasive medical device is provided. The device comprises an elongated member with proximal and distal ends, an inner radially expandable body surrounding the distal end of the elongated member, a stent surrounding the inner body, and an outer radially expandable body surrounding the stent. Optionally, the medical device further comprises a haemostatic coating surrounding the outer body. A method for treating tissue is also provided. The method comprises placing an elongated device into the tissue to create a path, radially expanding the device against the tissue to radially displace the tissue along the path, radially reinforcing the device against the tissue, and introducing a medical element through the device to perform a medical procedure on the tissue that can be image guided.

### FIELD OF THE INVENTION

The invention relates to medical devices, and in particular, medical devices for accessing tissue and creating access pathways in tissue to a target site.

### BACKGROUND OF THE INVENTION

Many medical procedures require access to deep brain tissue. For instance, it is known to surgically treat neurodegenerative diseases, such as Tumors, Alzheimer's Disease, Parkinson's Disease, Tremor, and Epilepsy, and ischemia of the brain, such as stroke, with procedures such as ablative surgery or restorative surgery.

Ablative and resection surgeries for tumor are common in the practice of neurosurgery for debulking a lesion for further adjunctive therapy or to alleviate pressure build up resulting from a mass in the skull. The tumor is removed using ablative therapies such as bipolar and laser, as well as resected with suction, excision or treatment with cavitronic ultrasound devices. The tumors are usually deep below the surface of the brain and normal brain tissue is often displaced to reach the target lesion with the assistance of significant localization technologies.

Some medical procedures for the treatment of stroke victims also require access to deep brain tissue. Strokes often leave clots in the brain called intracerebral hematomas. These hematomas can be removed by highly invasive surgery. They can also be removed by drainage after minimally invasive surgery. Once stabilized, the offending source or cause of the hematoma is addressed.

Although current surgical techniques have proven successful in the treatment of brain disorders, such techniques are still quite invasive, requiring access to deep brain tissue. Often, more superficial brain tissues are sacrificed while accessing deeper tissues. Typical devices used to gain access to deep tissue targets include large obturators with sharp piercing tips that cut superficial brain tissues overlying the deeper tissue targets. Cutting superficial brain tissues can cause significant damage. The trajectory is often calculated and guided with image based navigation systems that fuse historical imaging data with surface markers on the patient, increasing accuracy of the pathway to the target, guiding the surgeon through treacherous territory.

Thus, there remains a need to provide improved methods, apparatus, kits, and systems for accessing deep brain tissue targets, while minimizing damage to superficial brain tissue overlying those targets and creating a blood free conduit through which the surgeon can visualize the target and then operate on it.

### SUMMARY OF THE INVENTION

In accordance with a first aspect of the present invention, a medical device comprises an elongated member, which may be, e.g., introduced through tissue. Preferably, the elongated member has a blunt tip in order to minimize tissue trauma. The medical device further comprises a first radially expandable body (e.g., an inflatable balloon) surrounding the distal end of the elongated member, and a stent surrounding the first body. In one preferred embodiment, the elongated member and first body can be removed from the device, thereby creating a working channel within the stent to allow medical instruments or medicaments to be placed through the stent. In this case, the stent is preferably plastically deformable to maintain the working channel created by the removal of the elongated member.

The medical device further comprises a second radially expandable body (e.g, an expandable annular balloon) surrounding the stent. The second expandable body can, e.g., provide a means for radially displacing tissue along the path. The medical device may optionally comprise a haemostatic coating surrounding the second body to, e.g., prevent or minimize bleeding of tissue.

In accordance with a second aspect of the present invention, a method of treating a brain is provided. The brain may have a disorder, e.g. a tumor (benign or malignant), Epilepsy, and Huntington's, or a brain injury or infarction, such as stroke or other vascular lesions. The method comprises inserting an elongated device into the brain to create a path. In one preferred method, an opening is created in the cranium and the elongated device is inserted through the opening. Preferably, the elongated device is inserted between tissue layers to minimize tissue trauma. The medical device will often be fitted with emitters to allow recognition and guidance by a navigation system. The method further comprises radially expanding the device to radially displace the tissue along the path, and introducing a medical element (e.g, an instrument and/or medicament) through the device to perform a medical procedure on the brain. After the medical procedure is completed, the device may optionally be radially contracted and removed from the brain if desired.

In accordance with a third aspect of the present invention, a method of treating tissue is provided. The method comprises providing an elongated device having a first radially expandable body, surrounded by a stent, which is surrounded by a second radially expandable body. The method further comprises placing the device into the tissue (e.g., between tissue layers) to create a path. The method may optionally comprise treating the tissue in contact with the device with a haemostatic compound to reduce bleeding. The method further comprises radially expanding the second expandable body against the tissue to radially displace the tissue along the path. The method further comprises radially expanding the first expandable body to radially expand the stent, radially contracting and removing the first expandable body to create a working channel through the stent, and then introducing a medical element (e.g., a medical instrument or medicament) through the channel to perform a medical procedure on the tissue. The second expandable body is then radially contracted, and the device removed from the tissue.

### BRIEF DESCRIPTION OF THE DRAWINGS

The drawings illustrate the design and utility of preferred embodiment(s) of the invention, in which similar elements are referred to by common reference numerals. In order to better appreciate the advantages and objects of the invention, reference should be made to the accompanying drawings that illustrate the preferred embodiment(s). The drawings, however, depict the embodiment(s) of the invention, and should not be taken as limiting its scope. With this caveat, the embodiment(s) of the invention will be described and explained with additional specificity and detail through the use of the accompanying drawings in which:

FIG. 1 is a side view of an invasive medical device constructed in accordance with a preferred embodiment of the present invention;

FIG. 2 is a cross sectional view, along the axis, of the distal end of the medical device of FIG. 1;

FIG. 3 is a cross sectional view, perpendicular to the axis, of the distal end of the medical device of FIG. 1;

FIGS. 4A-4G are cross sectional views, along the axis of the medical device, illustrating a method of treating a tissue using the medical device of FIG. 1; and

FIGS. 5A-5G are cross sectional views, perpendicular to the axis of the medical device, illustrating a method of treating a tissue using the medical device of FIG. 1.

### DETAILED DESCRIPTION OF THE PREFERRED EMBODIMENTS

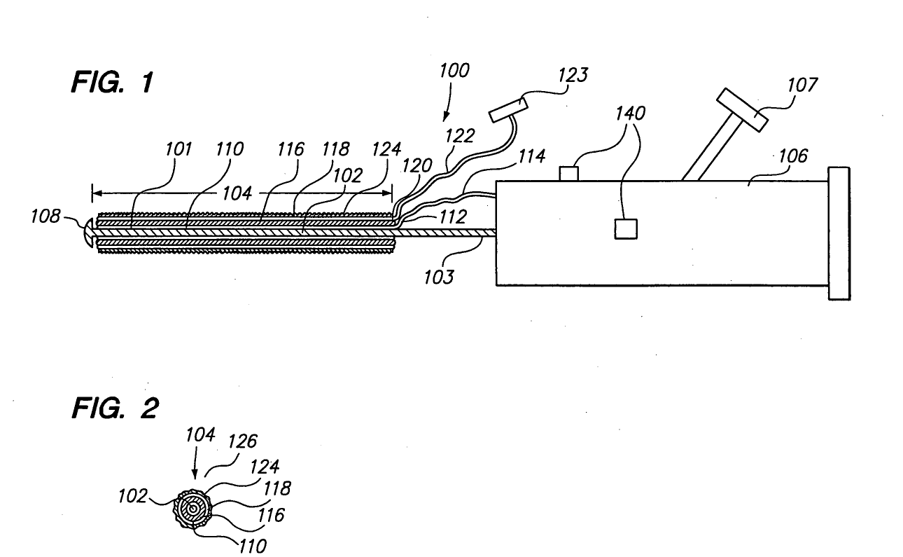

Referring to FIG. 1, a medical device 100 constructed in accordance with one preferred embodiment of the present invention is shown. In its simplest form, the device 100 comprises an elongated guide member 102 having a distal end 101 and a proximal end 103, a tissue channel forming assembly 104 mounted to the distal end 101 of the guide member 102, and a handle 106 mounted to the proximal end 103 of the guide member 102. The handle 106 can be ergonomically designed to allow a physician to more easily manipulate the device 100, and optionally features an injection port 107. Two emitters 140, that are separated from each other, are mounted onto the medical device 100 to allow guidance by a navigation system (not shown).

As will be described in greater detail below, the channel forming assembly 104 is configured to open a working channel in tissue through which surgical or diagnostic medical instruments or therapeutic agents can be introduced to reach a remote target tissue site. The tissue channel forming assembly 104 comprises two inflation tubes 114 and 122 through which an inflation medium is conveyed in order to actuate the channel forming assembly 104 in a manner described below. The first inflation tube 114 is connected to an inflation port 107 located on the handle 106, and the second inflation tube 122 is connected to a free inflation port 123. Alternatively, the first inflation tube 114 can be connected to a free inflation port (not shown), in which case, the inflation port 107 on the handle 106 will not be needed.

The guide member 102 can be composed of any suitable axially rigid or semi-rigid material, such as stainless steel, nitinol, etc. that allows the guide member 102 to be introduced through solid tissue. In this embodiment, the guide member 102 has a blunt tip 108 to minimize tissue damage during insertion. The length of the channel forming assembly 104, is preferably sized to approximate the length of the working channel that is to be formed through the tissue to reach the remote tissue target site. For example, exemplary lengths within the range of four to eight inches are typical. If the length of the channel forming assembly 104 is significantly greater than the length of the working channel, the difference in resistance to expansion may cause uneven expansion along the length of the channel forming assembly 104. The diameter of the guide member 102 is preferably equal to the minimum diameter necessary to maintain axial rigidity of the guide member 102. For example, exemplary diameters within the range of ten thousandths to forty thousandths of an inch are typical.

Referring to FIGS. 1 and 2, the channel forming assembly 104 will now be described in further detail. The channel forming assembly 104 is shown in FIGS. 1 and 2 as being in a low-profile fully collapsed state to aid in delivering the medical device 100 through the tissue. As will be described later, the channel forming assembly 104 can be placed into various stages of expanded states in order to create a working channel through tissue. To this end, the channel forming assembly 104 generally comprises an inner radially expandable body 110 surrounding the distal end 101 of the guide member 102, a stent 116 surrounding the inner body 110, and an outer radially expandable body 118 surrounding the stent 116.

In the illustrated embodiment, the inner body 110 takes the form of a balloon. The inner body 110 is preferably composed of a non-compliant or semi-compliant material, such as those typically used for angioplastly balloons. The inner body 110 may be formed of a wide variety of suitable compliant or non-compliant materials known in the art. However, for purposes of the present invention, elastomeric polymers are preferred. Examples of suitable elastomers include silicone, latex, and thermoplastic polyolefin rubbers. Alternatively, the inner body 110 may be formed of a thermoplastic polyisoprene rubber such as hydrogenated polyisoprene. Thermoplastic polyisoprene rubber has a number of advantages in terms of both performance and manufacture over conventional elastomeric materials.

For example, silicone balloons tend to yield larger profiles due to manufacturing limitations associated with wall thickness. In addition, silicone balloons are expensive to manufacture and assemble, because they require specialized manufacturing equipment and are not easily bonded to conventional shaft materials. Similarly, latex balloons are difficult to bond to conventional shaft materials. Latex balloons are considered toxic and excessively compliant. Balloons formed of thermoplastic polyolefin rubbers typically have a larger profile due to manufacturing limitations associated with wall thickness. Specifically, thermoplastic polyolefin rubbers usually contain a dispersion of Ethylene Propylene Diene Monomer (EPDM) rubber, which limits how thin the balloon tubing may be extruded.

By contrast, balloons formed of thermoplastic polyisoprene rubber, such as hydrogenated polyisoprene have superior performance and manufacturing attributes. For example, hydrogenated polyisoprene, which is commercially available under the trade name CHRONOPRENE from CT Biomaterials, may be processed with standard polyolefin processing equipment to obtain balloon tubing having a wall thickness of approximately 0.003 inches to 0.010 inches and a corresponding inside diameter of approximately 0.016 inches to 0.028 inches. Such balloon tubing has been demonstrated to produce balloons having a nominal outside diameter when inflated of approximately 3.0 mm to 5.5 mm. The wall thickness of the balloon is on the order of 0.001 inches, which allows the balloon to have a very low deflated profile, which in turn allows for insertion of into tissue with minimal damage.

Balloons made from thermoplastic polyisoprene rubber inflate uniformly and typically form a cylindrical shape when inflated. The rupture pressure has been shown to be approximately one atmosphere, which is desirable for creating working channels in tissue. The thermoplastic polyisoprene rubber has also demonstrated superior manufacturing capabilities. Hydrogenated polyisoprene is readily bondable to conventional shaft materials and may be extruded using conventional extrusion equipment.

The inner body 110 can be chemically bonded to the elongated member 102 with an adhesive or the two elements can be heat bonded together. The inner body 110 defines a first port 112 connected to the first inflation tube 114, which in turn may be connected to a fluid source, such as a syringe for inflating and deflating the inner body 110. Fluid introduced into the first inflation tube 114 will travel through the first port 112 and into the inner body 110, thereby placing the inner body 110 into its expanded geometry. Fluid removed from the first inflation tube 114 will, in turn, remove the fluid from the inner body 110, thereby placing the inner body 110 into its collapsed geometry.

Preferably, the stent 116 has a complex geometry allowing it to be packed into a low profile collapsed state and stiff and stable enough radially, in an expanded state, to maintain patency of the working channel. The stent 116 may consist of any biocompatable material possessing the structural and mechanical attributes necessary for supporting the working channel. Thus, both metallic and polymeric materials are suitable. Examples of preferred biocompatable metallic materials include stainless steel, tantalum, nitinol, and gold. Preferred polymeric materials may be selected from the list immediately below, which is not exhaustive: poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), polyD,L-lactide-co-caprolactone) (PLA/PCL), poly(glycolide-co-caprolactone) (PGA/PCL), polyanhydrides (PAN), poly(ortho esters), poly(phoshate ester), poly(amino acid), poly(hydroxy butyrate), polyacrylate, polyacrylamid, poly(hydroxyethyl methacrylate), elastin polypeptide co-polymer, polyurethane, polysiloxane and their copolymers.

The skeletal framework of the stent 116 may be formed through various methods as well. The framework may be welded, molded, or consist of filaments or fibers that are wound or braided together in order to form a continuous structure. The stent 116 is not bonded to the inner body 110, so that they may slide relative to each other along the longitudinal axis of the medical device 100. Once the stent 116 is expanded, it has sufficient strength to resist the inward pressure exerted by the tissue.

In this embodiment, the outer expandable body 118 takes the form of an annular balloon. The outer body 118 can be made of the same material as the inner body 110. The outer body 118 can be chemically bonded to the stent 116 with an adhesive or the two elements can be heat bonded together.

The outer body 118 defines a second port 120 connected to the second inflation tube 122, which in turn may be connected to a fluid source such as a syringe for inflating and deflating the outer body. Fluid introduced into the second inflation tube 122 will travel through the second port 120 and into the outer body 118, thereby placing the outer body 118 into its expanded geometry. Fluid removed from the second inflation tube 122 will, in turn, remove the fluid from the outer body 118, thereby placing the outer body 118 into its collapsed geometry. Because the first inflation tube 114 and the second inflation tube 122 are not connected to each other, the inner body 110 and outer body 118 can be expanded independently.

In the illustrated embodiment, the outer body 118 is surrounded by a haemostatic coating 124, which reduces bleeding when the device 100 is introduced through the tissue. The coating 124 can be a polymer that mechanically reduces bleeding. Alternatively, the coating 124 can contain a medicament that reduces bleeding, such as a vasoconstrictor or a coagulant.

In alternative embodiments, the medical device 100 does not comprise a outer body 118, in which case, the haemostatic coating 124 may surround the stent 116.

As can be appreciated from FIG. 2, the haemostatic coating 124, outer radially expandable body 118, stent 116, inner radially expandable body 110, and elongated member 102 completely surround each other. In alternative embodiments, the coating 124 does not completely surround the outer body 118, but sufficiently surrounds the outer body 118 to reduce bleeding from the tissue through which the medical device 100 is inserted. Similarly, in alternative embodiments, the outer body 118 does not completely surround the stent 116, but sufficiently surrounds the stent 116 to create a space between the tissue and the stent 116. In yet other alternative embodiments, the stent 116 does not completely surround the inner body 110, but sufficiently surrounds the inner body 110 to resist inward pressure exerted by the tissue. In other alternative embodiments, the inner body 110 does not completely surround the elongated member 102, but sufficiently surrounds the elongated member 102 to exert pressure to expand the stent 116.

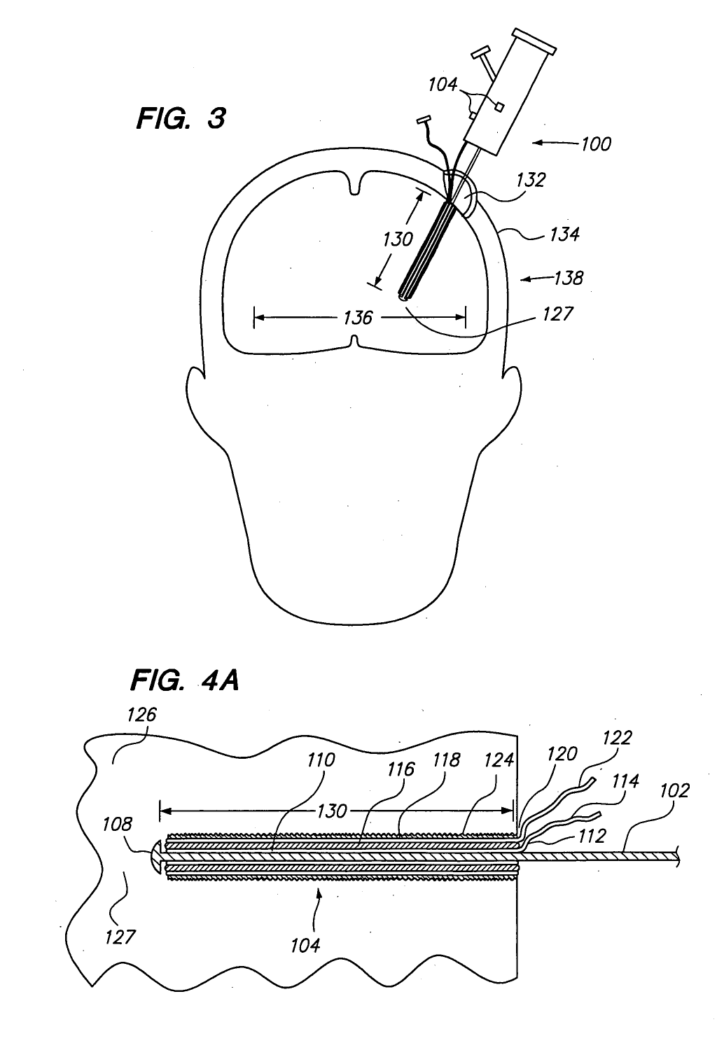

Having described the structure of the medical device 100, a method of using the medical device 100 to treat a remote tissue site 127, and in particular, a target site within the deep brain region 136 of a patient 138, will now be described with reference to FIGS. 3, 4A-4G and 5A-5G.

First, a burr hole 132 is created within the cranium 134 of the patient 138 using standard techniques known in the art (FIG. 3). Then, the guide member 102, with the channel forming assembly 104 in its low profile and fully collapsed state, is introduced through the burr hole 132 and inserted through tissue 126 to create a path 130 to the remote tissue target site 127 (FIGS. 3, 4A and 5A). Two emitters 140, mounted on the device allows medical device 100 to be guided by a navigation system (not shown). To minimize tissue trauma, the guide member 102 is preferably introduced between naturally occurring tissue planes (not shown), which in the case of brain tissue, take the form of spaces between axons, which can be enlarged with minimal damage to the axons or neurons of which they are a part. As the channel forming assembly 104 is inserted into the tissue 126, the haemostatic coating 124 both minimizes bleeding from the tissue 126 and lubricates the path 130 to reduce frictional shearing forces during insertion.

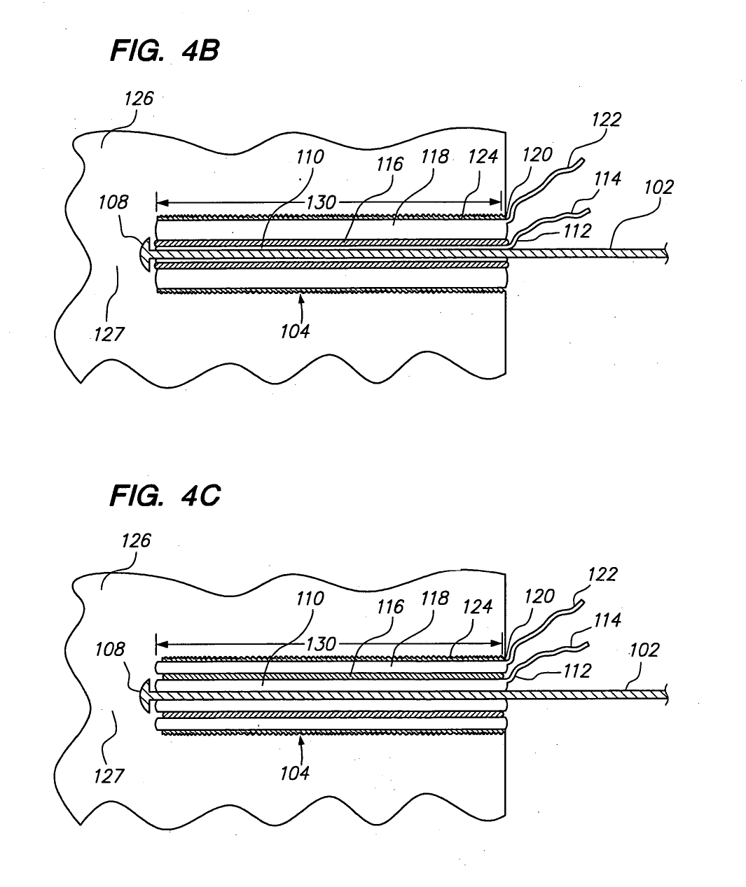

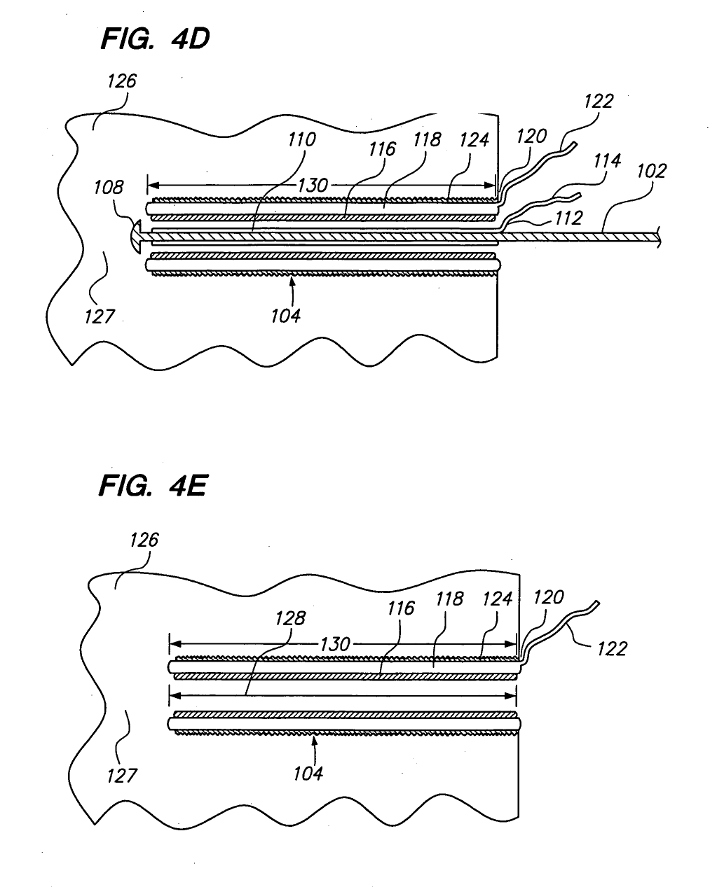

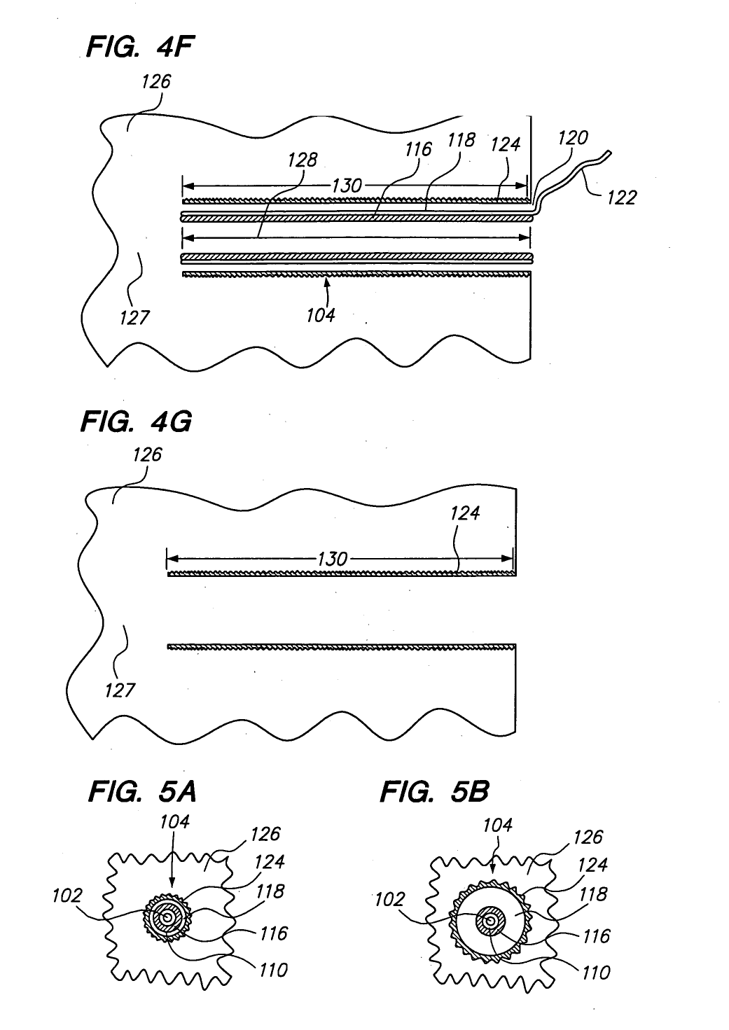

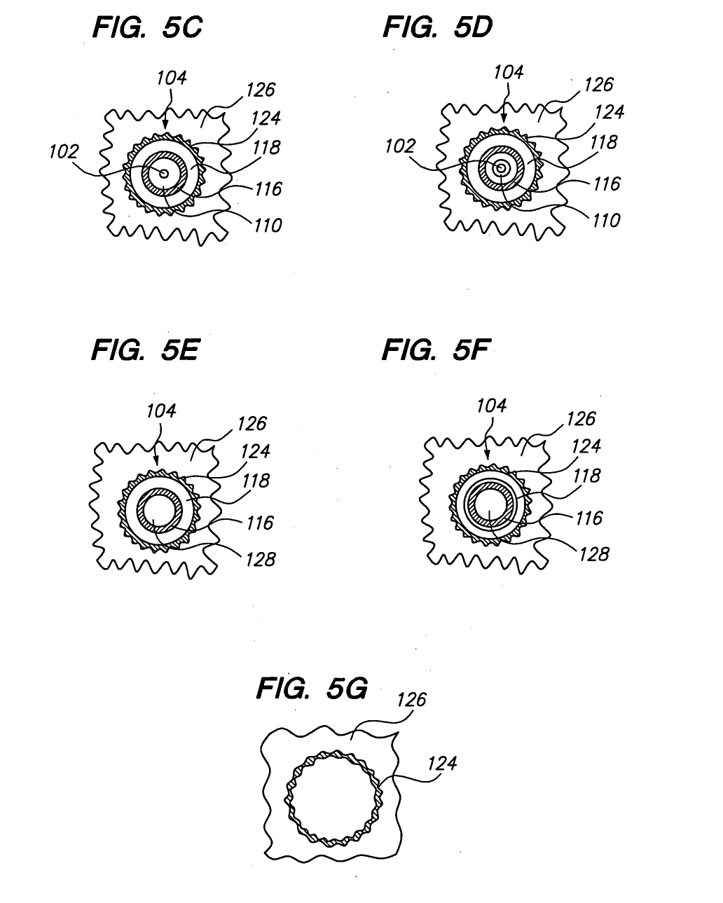

Once the channel forming assembly 104 is properly placed within the tissue 126, the outer body 118 is expanded to radially displace the brain tissue along the path 130 by conveying an inflation medium through the second inflation tube 122 and into the outer body 118 (FIGS. 4B and 5B). The outer body 118 may be incrementally expanded to prevent overly expanding the channel forming assembly 104 and damaging the tissue 126, e.g., by adding predetermined volumes of fluid. The outer body 118 may optionally be expanded by filling it with a coolant, thereby reducing the temperature of the tissue 126 and further minimizing blood loss from the tissue 126.

After the outer body 118 has been expanded, the inner body 110 is expanded by conveying an inflation medium through the first inflation tube 114 located in the handle 106 (shown in FIG. 1) and into the inner body 110 (FIGS. 4C and 5C), which in turn, expands the stent 116, thereby reinforcing the outer body 118 against the displaced tissue 126. The inner body 110 may be incrementally expanded to prevent overly expanding the channel forming assembly 104 and damaging the tissue 126, e.g., by adding predetermined volumes of the inflation medium (air or biocompatable fluid).

During this step, the outer body 118 may be allowed to partially collapse by releasing fluid through the second port 120 into the second inflation tube 122, while the inner body 110 is expanded to reduce the pressure needed to expand the inner body 110 and the stent 116.

After the stent 116 is expanded to reinforce the radially displaced tissue, the inner body 110 is collapsed (FIGS. 4D and 5D) by releasing fluid through the first inflation tube 114 and out of the inflation port 107 on the handle 106 (both shown in FIG. 1). Because the stent 116 expands plastically, it maintains the patency of the working channel 128 despite the collapsing of the inner body 110 Once the inner body 110 is collapsed, the inner body 110 and the elongated member 102 are removed to form a working channel 128 through the stent 116. (FIGS. 4E and 5E) A medical element (not shown) is then introduced into the working channel 128 to perform a medical procedure on the tissue target site 127. Medical procedures that may be performed through the working channel 128 include ablative surgeries, restorative surgeries, and chemotherapy. Medical elements that may be introduced through the working channel 128 include ablation probes, needles, scalpels, and medicaments, such as chemotherapy agents. Diagnostic procedures can also be performed through the working channel 128.

After the medical procedure has been performed, the outer body 118 is collapsed by releasing fluid through the second inflation tube 122 and out of the inflation port 123 (shown in FIG. 1). Once the outer body 118 is collapsed, it is removed from the working channel 128 (FIGS. 4F and 5F). Collapsing the outer body 118 reduces the profile of the tissue channel forming assembly 104 and allows it to be removed from the tissue 126 with minimal frictional shearing forces on the tissue 126. During removal of the tissue channel forming assembly 104, the haemostatic coating 124 acts as a lubricant to further minimize frictional shearing forces on the tissue 126. The haemostatic coating 124 is retained in the tissue 126 (see FIGS. 4G and 5G) to medically minimize bleeding from the tissue 126.

Tissues that may be suitably treated using the above-described method include brain, liver, or any other tissue forming a solid organ.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

1. A medical device, comprising:
an elongated member having a proximal end and a distal end;
a first radially expandable body surrounding the distal end of the member;
a stent surrounding the first body, wherein radial expansion of the first body radially expands the stent; and
a second radially expandable body surrounding the stent, wherein the first and second bodies are independently expandable.

2. The device of claim 1, wherein the distal end of the member is blunted.

3. The device of claim 1, wherein the first and second bodies are balloons.

4. The device of claim 1, wherein the first and second bodies expand elastically.

5. The device of claim 1, wherein the stent expands plastically.

6. The device of claim 1, wherein the second body is an annular balloon.

7. The device of claim 1, further comprising a haemostatic coating surrounding the second body.

8. The device of claim 1, wherein the first and second bodies and the stent extend along a majority length of the member.

9. The device of claim 1, wherein the elongated member and the first body are removable from the device.

10. The device of claim 1, wherein the elongated member is axially rigid.

11. The device of claim 1, further comprising an emitter, configured to send navigational signals.

12. A method of treating a brain, comprising:
inserting an elongated device into the brain to create a path;
radially expanding the device to create a working channel along the path; and
introducing a medical element into the working channel to perform a medical procedure on the brain.

13. The method of claim 12, wherein the medical element is an instrument.

14. The method of claim 12, wherein the medical element is a medicament.

15. The method of claim 12, further comprising creating an opening in a cranium, and inserting the elongated device through the opening.

16. The method of claim 12, further comprising inserting the elongated device between tissue layers within the brain.

17. The method of claim 12, further comprising radially contracting and removing the device from the brain.

18. A method of treating a tissue, comprising:
placing an elongated device into the tissue to create a path;
radially expanding the device against the tissue to radially displace the tissue along the path;
radially reinforcing the device against the tissue; and
introducing a medical element through the device to perform a medical procedure on the tissue.

19. The method of claim 18, wherein the medical element is an instrument.

20. The method of claim 18, wherein the medical element is a medicament.

21. The method of claim 18, further comprising inserting the elongated device between tissue layers.

22. The method of claim 18, further comprising radially contracting and removing the device from the tissue.

23. The method of claim 18, further comprising treating the tissue in contact with the device with a haemostatic compound to reduce bleeding.

24. A method of treating a tissue, comprising:
placing an elongated device, having a first radially expandable body, surrounded by a stent, which is surrounded by a second radially expandable body, into the tissue to create a path;
radially expanding the second expandable body against the tissue to radially displace the tissue along the path;
radially expanding the first expandable body to radially expand the stent to reinforce the second expandable body against the tissue;
radially contracting and removing the first expandable body to create a working channel through the stent;
introducing a medical element through the channel to perform a medical procedure on the tissue; and
radially contracting the second expandable body and removing the device from the tissue.

25. The method of claim 24, wherein the medical element is an instrument.

26. The method of claim 24, wherein the medical element is a medicament.

27. The method of claim 24, further comprising inserting the elongated device between tissue layers.

28. The method of claim 24, further comprising treating the tissue in contact with the device with a haemostatic compound to reduce bleeding.
